(19) United States
(12) Patent Application Publication
(10) Pub. No.: US 2017/0065152 A1
(43) Pub. Date: Mar. 9, 2017

(54) ENDOSCOPE

(71) Applicant: OLYMPUS CORPORATION, Tokyo (JP)

(72) Inventor: Reiji KOYAMA, Tokyo (JP)

(73) Assignee: OLYMPUS CORPORATION, Tokyo (JP)

(21) Appl. No.: 15/355,119

(22) Filed: Nov. 18, 2016

Related U.S. Application Data
(63) Continuation of application No. PCT/JP2015/075882, filed on Sep. 11, 2015.

(30) Foreign Application Priority Data
Sep. 16, 2014 (JP) 2014-188040

(51) Int. Cl. A61B 1/005; A61B 1/06; A61B 1/04; A61B 1/00; A61B 1/018

(52) U.S. Cl. CPC A61B 1/0052; A61B 1/00112; A61B 1/018; A61B 1/0057; A61B 1/04; A61B 1/0661; G02B 23/26

(57) ABSTRACT

An endoscope includes: an insertion portion configured to be inserted into a subject; a plate-like member provided on a proximal end side of the insertion portion, the plate-like member allowing a mechanism component for operating the insertion portion to be arranged on the plate-like member, the plate-like member having a predetermined thickness dimension; an attachment portion formed by flexing an end portion of the plate-like member in a predetermined direction; and a fixing portion configured to fix at least part of another member within a range of the thickness of the plate-like member, the fixing portion being formed inside the attachment portion.

### CROSS REFERENCE TO RELATED APPLICATION

This application is a continuation application of PCT/JP2015/075882 filed on Sep. 11, 2015 and claims benefit of Japanese Application No. 2014-188040 filed in Japan on Sep. 16, 2014, the entire contents of which are incorporated herein by this reference.

### BACKGROUND OF THE INVENTION

1. Field of the Invention

The present invention relates to an endoscope including a partition plate configured to divide an internal space of an operation portion in two.

2. Description of the Related Art

In recent years, insertion apparatuses to be inserted into a subject/object, for example, endoscopes, have been used in a medical field and an industrial field. An elongated insertion portion of an endoscope used in the medical field is inserted into a body. An elongated insertion portion of an endoscope used in the industrial field is inserted into, e.g., an inner portion of a jet engine or the inside of a piping of a plant.

An endoscope includes, e.g., bending wires, various fluid tubes, a light guide fiber bundle and a signal cable as incorporated components of the endoscope.

The incorporated components of the endoscope that are inserted inside an insertion portion are introduced in an internal space of an operation portion. In order to downsize the operation portion, it is necessary to use the internal space effectively. The inside of the operation portion is partitioned by a baseplate, which is a partition plate.

On one surface side of the baseplate, the tubes and the like from among the incorporated components are arranged, and on the other side of the baseplate, for example, four bending wires are arranged. As illustrated in FIG. 2 in Japanese Patent Application Laid-Open Publication No. 2008-092968, the bending wires are joined respectively to end portions of an up/down chain and end portions of a left/right chain, which correspond to bending directions, via respective joining members arranged on a fixed plate, which is a baseplates, provided inside the operation portion.

Note that, if the baseplate allows components to be attached not only to front and back faces on the one and other sides but also to side faces of the baseplate, the internal space of the operation portion can be effectively used, which is effective for downsizing the operation portion.

The baseplate is, for example, an aluminum plate and is formed in a desired shape by means of die casting. In each of one surface, the other surface and side faces of the baseplate, e.g., a plurality of screw holes configured to threadably connect members to the baseplate via screws are provided.

### SUMMARY OF THE INVENTION

An endoscope according to an aspect of the present invention includes: an insertion portion configured to be inserted into a subject; a plate-like member provided on a proximal end side of the insertion portion, the plate-like member allowing a mechanism component for operating the insertion portion to be arranged on the plate-like member, the plate-like member having a predetermined thickness dimension; an attachment portion including flexed portions formed by flexing an end portion of the plate-like member in a predetermined direction; an opening portion provided in the plate-like member, the opening portion being configured to open adjacent to the attachment portion; and a fixing portion configured to fix at least part of another member within a range of the thickness of the plate-like member, the fixing portion being formed inside the attachment portion.

An endoscope according to an aspect of the present invention includes: an insertion portion configured to be inserted into a subject; a plate-like member provided on a proximal end side of the insertion portion, the plate-like member allowing a mechanism component for operating the insertion portion to be arranged on the plate-like member, the plate-like member having a predetermined thickness dimension; an attachment portion formed by flexing an end portion of the plate-like member in a predetermined direction; an end face of the attachment portion, the end face being formed toward a direction opposite to the predetermined direction and protruding in the direction opposite to the predetermined direction relative to one flat surface of the plate-like member, the one flat surface being formed toward the direction opposite to the predetermined direction; and a fixing portion configured to fix at least part of another member within a range of the thickness of the plate-like member, the fixing portion being formed inside the attachment portion.

### BRIEF DESCRIPTION OF THE DRAWINGS

FIGS. 1 and 2 are diagrams for describing an overall configuration of an endoscope according to the present invention, and FIG. 1 is a one-side view illustrating a side face on the bending operation apparatus side;

FIG. 2 is the other-side view illustrating a side face on the side opposite to the bending operation apparatus side;

FIG. 3 is a diagram for describing a baseplate configured to divide an internal space of an operation portion of the endoscope in two;

FIG. 4 is a diagram for describing tubes and the like disposed on the second space side formed as a result of the division by the baseplate;

FIG. 5 is a diagram for describing a bending operation mechanism disposed on one surface side of a main frame;

FIG. 6 is a diagram for describing a main frame, the diagram including an enlarged view illustrating a unit fixing portion;

FIG. 7A is a diagram for describing a plate-like member before press working and a main frame subjected to press working;

FIG. 7B is a diagram for describing the plate-like member before press working and the main frame subjected to press working;

FIG. 7C is a front view of the unit fixing portion illustrated in FIG. 7B as viewed in the arrow Y7B direction;

FIG. 8 is a cross-sectional view along arrow Y8-Y8 in FIG. 7C;

FIG. 9 is a cross-sectional view along arrow Y9-Y9 in FIG. 7C;

FIG. 10 is a diagram indicating a height of the unit fixing portion of the main frame subjected to press working and a height of a baseplate shaped by means of die casting; and

FIG. 11 is a diagram for describing a main frame and, e.g., a bending operation mechanism fixedly provided on the main frame.

### DETAILED DESCRIPTION OF THE PREFERRED EMBODIMENT(S)

The present invention will be described below using an illustrated embodiment.

In each of the drawings used for the description of the present invention, respective components may be illustrated on difference scales so that the respective components have sizes that are large enough to be recognized in the drawing. Therefore, in the present invention, the counts and amounts of the components, the shapes of the components, and the size ratios and the relative positional relationships among the respective components illustrated in the drawings are not limited only to those illustrated in the drawings.

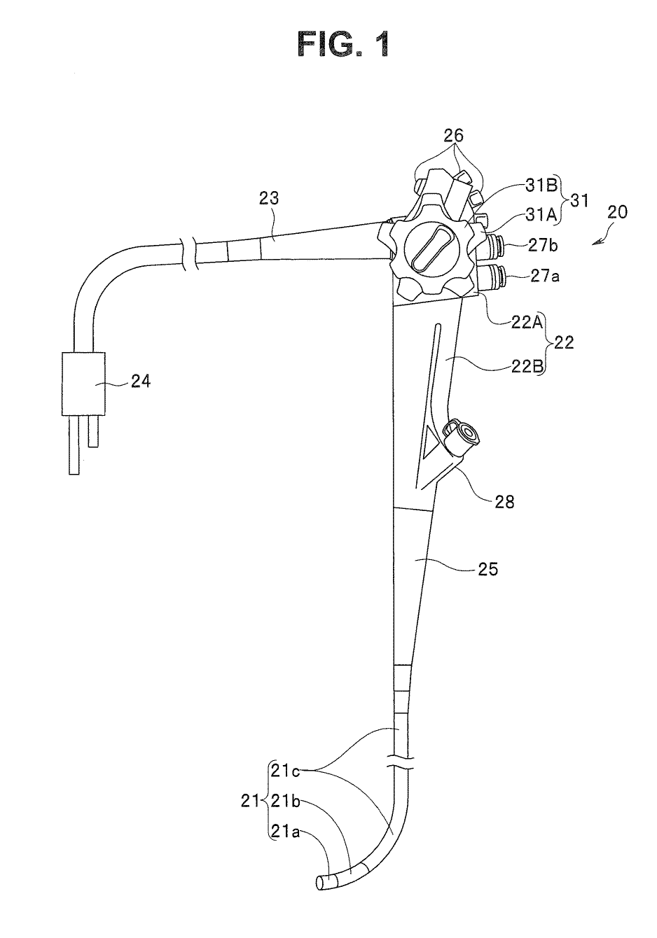

An overview of an overall configuration of an endoscope according to the present invention will be described with reference to FIGS. 1 to 4.

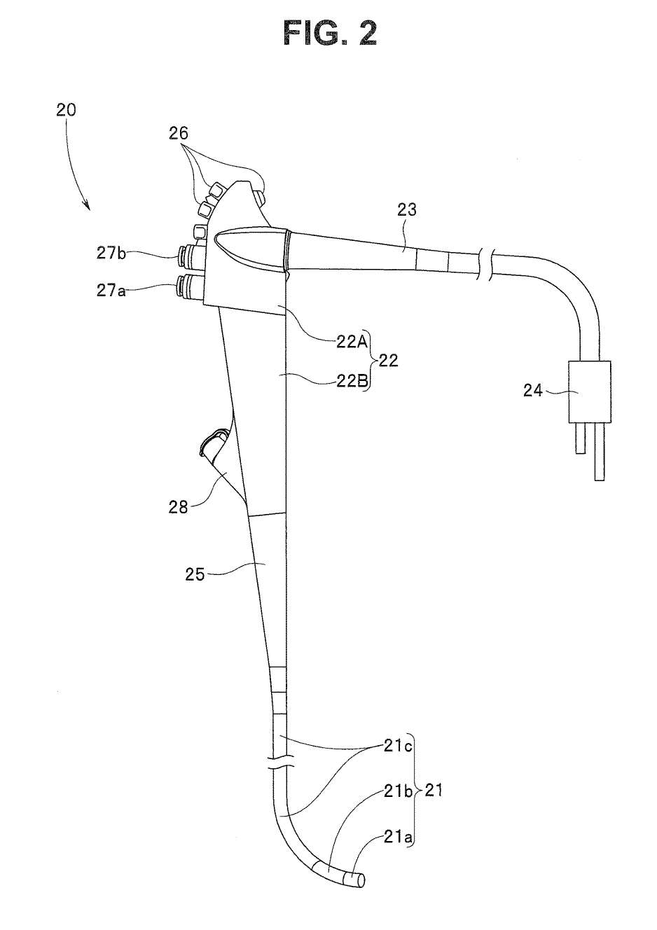

As illustrated in FIGS. 1 and 2, an endoscope 20 mainly includes e.g., an elongated insertion portion 21 to be inserted into, for example, a body cavity, which is a subject, an operation portion 22 provided so as to be continuous with the proximal end side of the insertion portion 21, a universal cord 23 extending from one side face of the operation portion 22, and a connector 24 disposed at an end portion of the universal cord 23.

The insertion portion 21 is configured by joining a rigid distal end configuration portion 21a, a bending portion 21b configured to bend in four directions and an elongated flexible tube portion 21c having flexibility in order from the distal end side. The bending portion 21b is capable of a bending motion in each of four directions, upward, downward, leftward and rightward, and also in an arbitrary direction by combining bending motions in the four directions.

Note that as an internal component unit for operating the bending portion 21b to bend, a bending operation mechanism 30 is provided in the operation portion 22. A specific configuration of the bending operation mechanism 30 will be described later.

In a distal end face of the distal end configuration portion 21a, although not illustrated, e.g., an objective lens, illumination lenses and a cleaning nozzle are provided and an opening of a treatment instrument channel is formed.

Inside the distal end configuration portion 21a, although not illustrated, e.g., an air feeding conduit and a water feeding conduit that are joined to the cleaning nozzle, and light guide fibers configured to supply illuminating light to the illumination lenses are disposed in addition to, e.g., an image pickup device, an electric substrate on which electric components are mounted and a video cable extending from the image pickup device.

The video cable and the light guide fibers extend to a connector 24 through the inside of the insertion portion 21, the operation portion 22 and the universal cord 23. Also, the air feeding conduit and the water feeding conduit are inserted through the insertion portion 21 and extend to the connector 24 via an air/water feeding cylinder provided in the operation portion 22 and the universal cord 23.

The operation portion 22 includes an operation portion body 22A and a grasping portion case body 22B integrally fixed in a water-tight manner. A proximal end portion of the insertion portion 21 is provided so as to be continuous with one end portion of the grasping portion case body 22B. Reference numeral 25 denotes a bend preventing portion. The bend preventing portion 25 prevents the flexible tube portion 21c of the insertion portion 21 from buckling. The bend preventing portion 25 is, e.g., an elastic rubber member, and covers a part of joining of the one end portion of the grasping portion case body 22B and the proximal end portion of the insertion portion 21.

At the operation portion body 22A of the operation portion 22, various operation members, for example, a plurality of electric switches 26 for remotely operating peripheral devices such as a video processor, an air/water feeding button 27a and a suction button 27b are provided. Also, at the operation portion body 22A, an upward/downward bending operation knob 31A and a leftward/rightward bending operation knob 31B, which are included in a bending operation apparatus 31, are rotatably disposed in order to operate the bending portion 21b of the insertion portion 21 to bend.

Reference numeral 28 denotes a treatment instrument introduction port, which is provided in the grasping portion case body 22B.

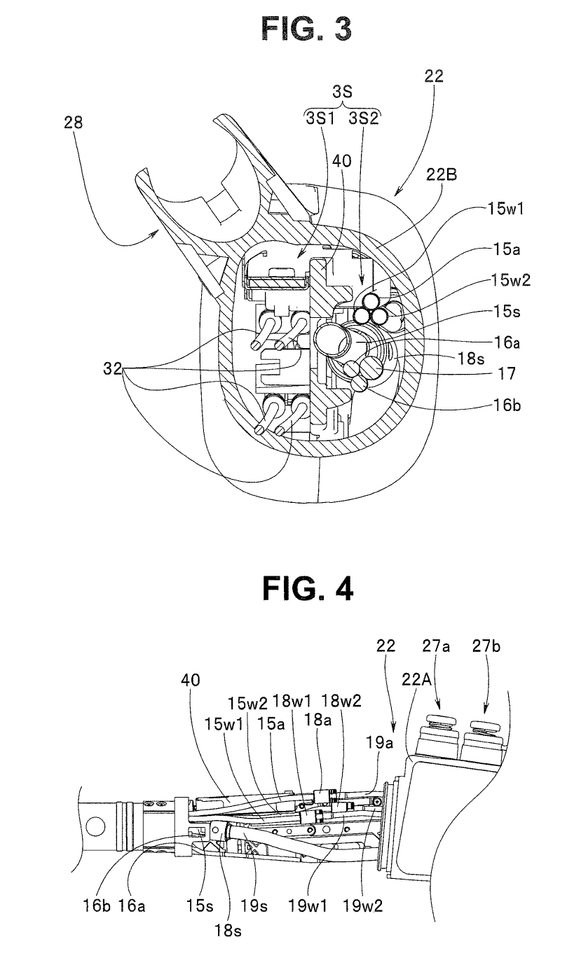

As illustrated in FIG. 3, incorporated components of the endoscope that are inserted inside the insertion portion 21 are introduced inside an operation portion internal space 3S, which is an internal space of the operation portion 22. The operation portion internal space 3S is divided in two spaces, which are a first space 3S1 and a second space 3S2, by a main frame 40, which is a baseplate. The main frame 40 is arranged in parallel with a longitudinal axis direction of the operation portion 22.

In the first space 3S1, four bending wires 32 corresponding to the bending directions of the bending portion 21b are introduced. The four bending wires 32 are an upward bending wire, a downward bending wire, a leftward bending wire and a rightward bending wire.

On the other hand, in the second space 3S2, an air feeding tube 15a, water feeding tubes 15w1, 15w2, a suction tube 15s that doubles as a treatment instrument channel tube, two fiber bundles 16a, 16b and a signal cable 17 are introduced.

Then, as illustrated in FIG. 4, one end of the air feeding tube 15a arranged on the one surface side of the main frame 40 is joined to a first tube 19a including one end joined to an air/water feeding button 5, via an air feeding tube joining fitting 18a, one end of the water feeding tube 15w1 is joined to a second tube 19w1 including one end joined to the air/water feeding button 27a, via a first water feeding tube joining fitting 18w1, one end of the water feeding tube 15w2 is joined to one end of a third tube 19w2 extending to an endoscope connector (not illustrated), via a second water feeding tube joining fitting 18w2, and one end of the suction tube 15s is joined to a fourth tube 19s including one end joined to the suction button 27b, via a suction tube joining fitting 18s.

The four bending wires 32 are introduced in the first space 3S1 and then joined respectively to end portions of an up/down chain and end portions of a left/right chain, which will be described later.

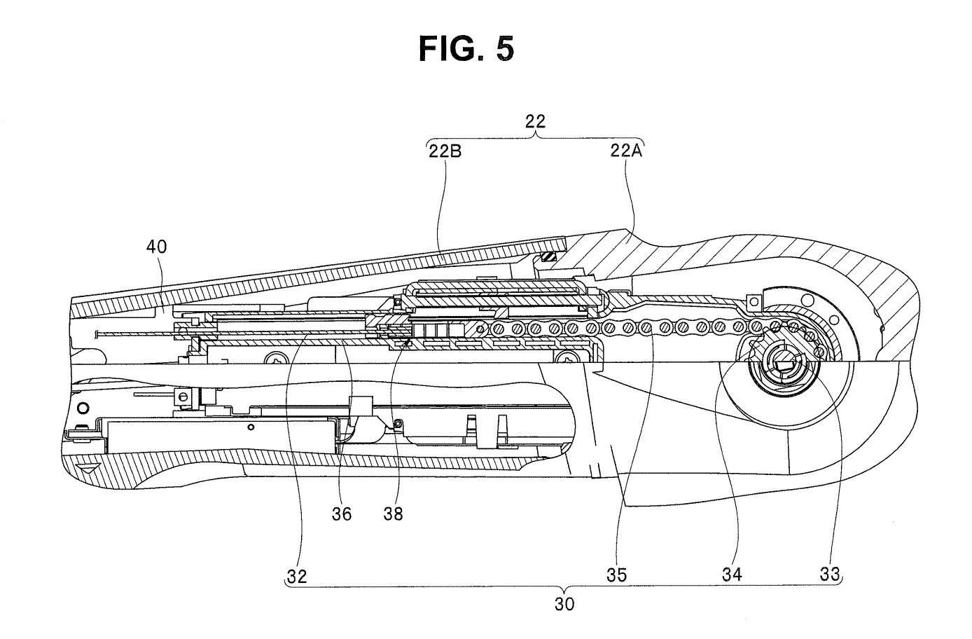

As illustrated in FIG. 5, the main frame 40, which is a baseplate, is fixed in an inner portion of the operation portion body 22A and an inner portion of the grasping portion case body 22B of the operation portion 22 by means of screw fastening. On the one surface side of the main frame 40, the bending operation mechanism 30 configured to pull/loosen the bending wires 32 to bend the bending portion 21b, a partition portion 36, and bending adjustment units 37 are disposed. The bending operation mechanism 30 mainly includes the bending operation apparatus 31 illustrated in FIG. 1, the bending wires 32 illustrated in FIG. 5, a spindle 33, sprockets 34 and chains 35.

Reference numeral 38 denotes a known joining member configured to join a bending wire 32 and a chain 35.

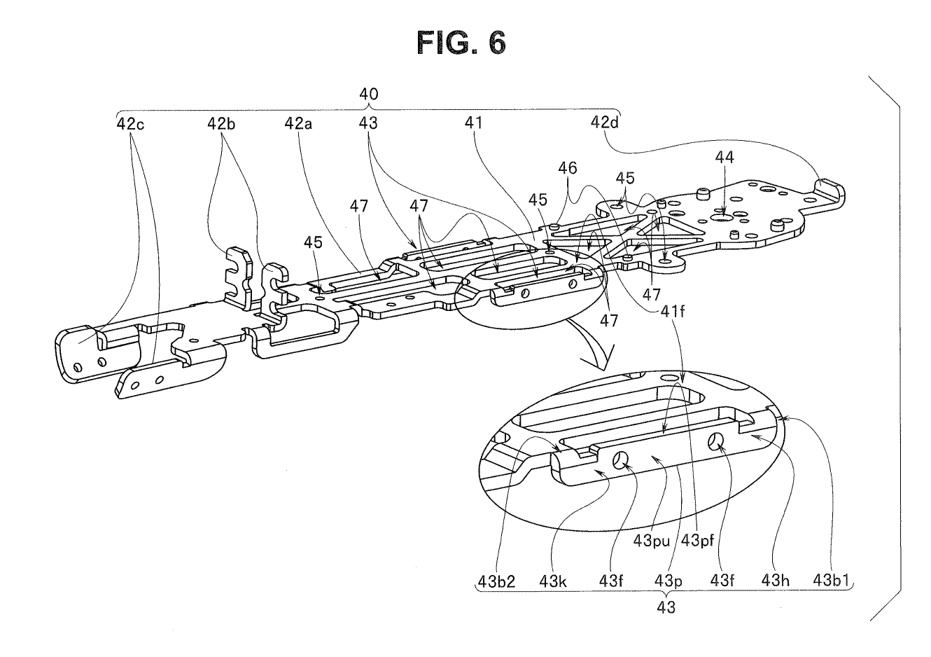

As illustrated in FIG. 6, the main frame 40 is formed in a predetermined shape resulting from subjecting a plate-like member of, for example, a stainless steel, the plate-like member having an elongated shape having a longitudinal axis and a predetermined thickness dimension, to press working.

In the main frame 40, a frame body 41, which is a planar portion, a plurality of folded portions 42a, 42b, 42c, 42d formed by means of folding relative to the frame body, and a pair of unit fixing portions 43 are provided.

Each unit fixing portion 43 includes a pair of flexed portions 43L1, 43L2, an attachment portion 43p, and, for example, two screw holes 43f, which are fixing portions. The fixing portions are not limited to the screw holes 43f and may be, e.g., protrusion portions.

The flexed portions 43L1, 43L2 are provided integrally with the frame body 41. The flexed portions 43L1, 43L2 are flexed and formed so that the attachment portion 43p is arranged in a predetermined direction relative to one flat surface 41f of the frame body 41. Then, the flexed portions 43L1, 43L2 each include an inner curved surface 43b1 having a small radius of curve and an outer curved surface 43b2 having a large radius of curve.

The attachment portion 43p is provided integrally with the flexed portions 43L1, 43L2. The attachment portion 43p includes an attachment surface 43pu to which, for example, a bending adjustment unit 37, which is another member, is attached. The attachment surface 43pu of the attachment portion 4-3p integrated with the flexed portions 43L1, 43L2 is arranged in a positional relationship in which the attachment surface 43pu is orthogonal to the one flat surface 41f of the frame body 41. Each of the flexed portions 43L1, 43L2 is an L-flexed portion having an L-shape. In other words, the attachment portion 43p includes the flexed portions 43L1, 43L2s provided integrally with the frame body 41, which is a plate-like member, the flexed portions 43L1, 43L2s being each foimed as a result of flexing an end portion of the frame body 41 in the predetermined direction. Note that the one flat surface 41f is a surface formed toward a direction opposite to the predetermined direction in the frame body 41. Furthermore, for stable attachment of the bending adjustment unit 37, the attachment portion 43p is formed so as to have a dimension that is larger than the predetermined thickness dimension of the frame body 41 in a direction orthogonal to the one flat surface 41f of the frame body 41.

The screw holes 43f are provided at respective predetermined positions in the attachment portion 43p. Male screws for fixing the bending adjustment unit 37 are threadably connected to the screw holes 43f.

A thickness of the attachment portion 4-3p, that is, the thickness of the main frame 40 is set so that female threads each including a thread ridge ensuring thread fixing via a male screw can be formed.

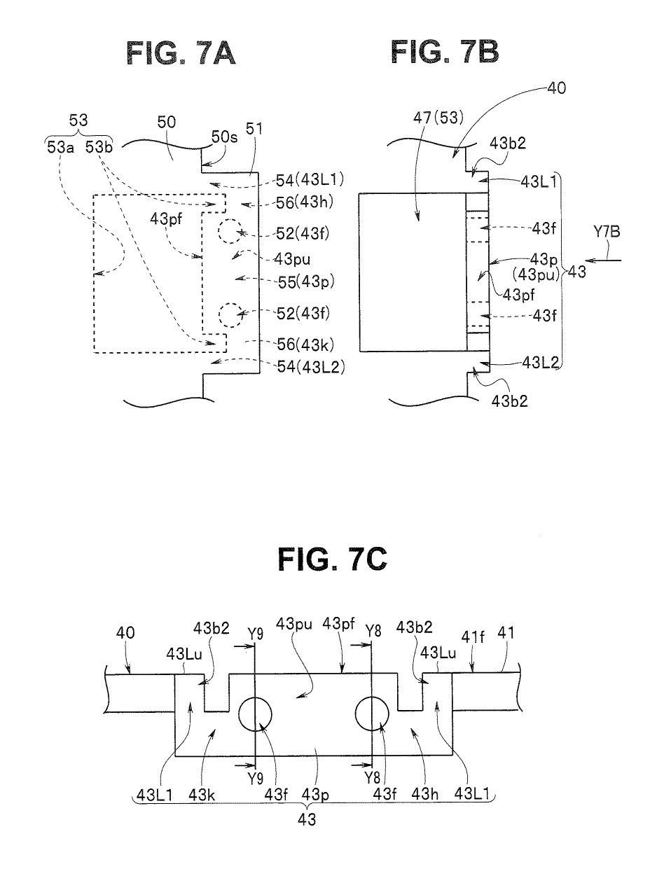

As illustrated in FIG. 7A, in a side portion of a plate-like member 50 having a predetermined thickness dimension, which is to be formed into a main frame 40, a unit fixing portion foaming portion 51, which is a protrusion portion protruding in a predetermined amount from a side face 50s, is provided.

In the unit fixing portion forming portion 51, prepared holes 52 for screw holes 43f, which are indicated by dash lines, and an opening portion 53 having a predetermined shape, which is indicated by dash lines, are formed by press working. The prepared holes 52 are subjected to female threading processing.

An opening of the opening portion 53 is provided adjacent to the attachment portion 43p. The opening of the opening portion 53 is shaped so as to include a rectangular hole 53a and a pair of cutout portions 53b. The rectangular hole 53a is provided so as to determine a position at which an opening-side end face 43pf of the attachment portion 43p is formed relative to the one flat surface 41f, and the rectangular hole 53a doubles as a through hole for weight reduction. Note that the opening-side end face 43pf is an end face of the attachment portion 43p, the end face being formed toward the direction opposite to the predetermined direction.

The pair of cutout portions 53b is provided to form a pair of flexed portion forming portions 54, an attachment portion forming portion 55 and a pair of connection portion forming portions 56 at respective predetermined positions in the unit fixing portion forming portion 51.

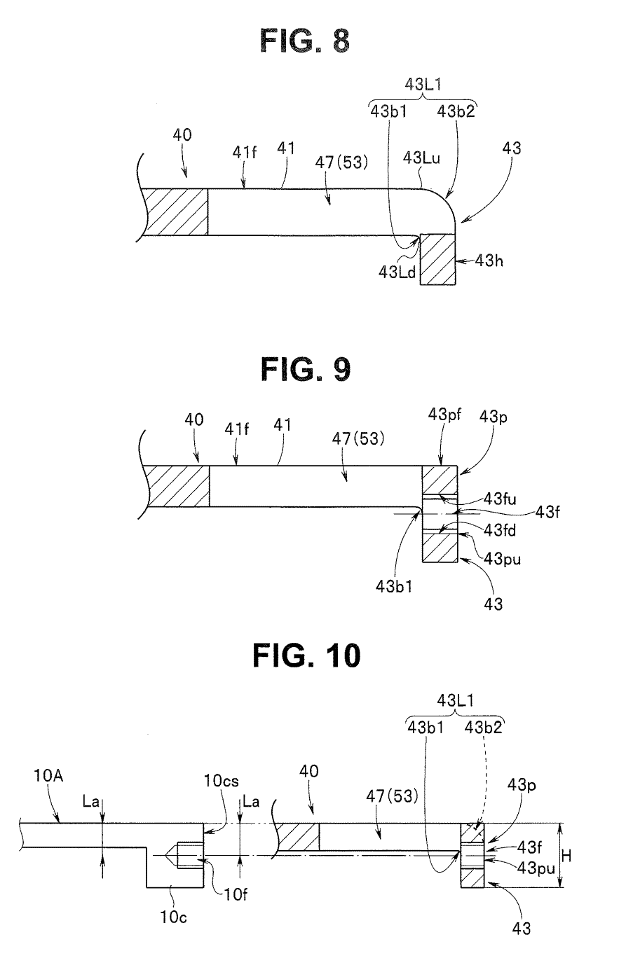

The aforementioned pair of flexed portion forming portions 54 of the unit fixing portion forming portion 51 is flexed into a predetermined state by press working and thereby is formed into a pair of flexed portions 43L1, 43L2 of a unit fixing portion 43, which is illustrated in FIGS. 7C, 8 and 9.

Simultaneously with the formation of the pair of flexed portions 43L1, 43L2, the attachment portion forming portion 55 is formed into an attachment portion 43p integrated with the pair of flexed portions 43L1, 43L2, the attachment portion 43p having a predetermined width dimension.

Here, an attachment surface 43pu of the attachment portion 43p is arranged in a positional relationship in which the attachment surface 43pu is orthogonal to one flat surface 41f. An opening-side end face 43pf of the attachment portion 43p protrudes to a predetermined level from the one flat surface 41f.

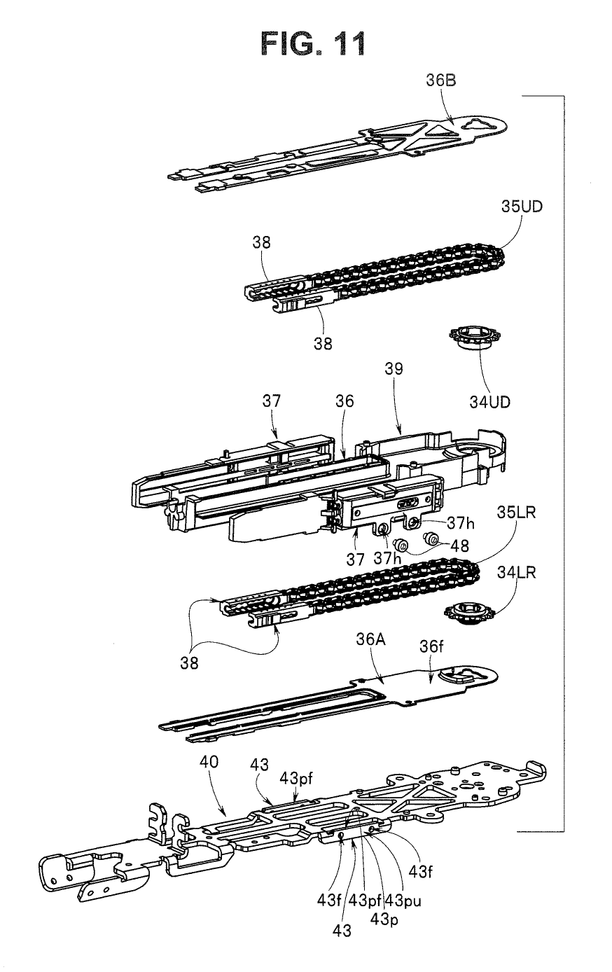

More specifically, the opening-side end face 43pf of the attachment portion 43p is arranged at a position at a same level as one flat surface 36f of a later-described first chain cover 36A, which is illustrated in FIG. 11, or a position that is lower than the flat surface 36f in a state in which the first chain cover 36A is mounted on the one flat surface 41f.

Simultaneously with the formation of the pair of flexed portions 43L1, 43L2, the pair of connection portion forming portions 56 is formed into a first connection portion 43h connecting to one flexed portion 43L1 and the attachment portion 43p integrally and a second connection portion 43k connecting the other flexed portion 43L2 and the attachment portion 43p integrally.

As described above, as a result of providing the unit fixing portion 43 in the main frame 40 by means of press working, as illustrated in FIG. 10, a height H of the unit fixing portion 43 of the main frame 40 is the same as a height of a baseplate 10A shaped by die casting.

Also, a distance from the one surface of the main frame 40 to a center axis of a screw hole 43f provided in the attachment portion 43p of the unit fixing portion 43 is equal to a distance La from an end face of the baseplate 10A to a center axis of a screw hole 10f formed in a protrusion portion 10c.

Consequently, an inexpensive main frame 40 manufactured using press working can be provided.

Furthermore, each screw hole 43f provided in the unit fixing portion 43 of the main frame 40 can be provided in a part of the attachment surface 43pu between an upper end 43Lu of an outer curved surface 43b2 and a lower end 43Ld of an inner curved surface 43b1 of each of the flexed portions 43L1, 43L2. In other words, it is possible that while an upper hole end portion 43fu of each screw hole 43f is arranged at a position that is higher than the lower hole end 43Ld of the inner curved surface 43b1, a lower end 43fd of each screw hole 43f is arranged at a position that is lower than the upper end 43Lu of the outer curved surface 43b2.

In other words, a region in which the screw holes 43f of the unit fixing portion 43 are provided can be set within a region at a same level as a region in which the flexed portions 43L1, 43L2 are provided, enabling effective use of the internal space for downsizing the operation portion.

In the frame body 41, a spindle hole 44, which is a through hole for fixedly providing the spindle 33, a plurality of screw holes 45 for thread connection fixing, a plurality of dowels 46 for attachment keeping and a plurality of through holes 47 for weight reduction are formed.

In the above-described embodiment, the opening-side end face 43pf of each attachment portion 43p projects to a predetermined level from the one flat surface 41f. The height of the projection of the opening-side end face 43pf can be adjusted by arbitrarily changing a shape of the opening. As a result of the shape of the opening being arbitrarily changed, the level of the opening-side end face 43pf can be made to, e.g., be the same as the level of the one flat surface 41f or be lower than the one flat surface 41f.

Also, as illustrated in FIG. 11, on the one surface of the main frame 40, the first chain cover 36A, a left/right sprocket 34LR and a left/right chain 35LR, a chain separator 39, an up/down sprocket 34UD and an up/down chain 35UD, a second chain cover 36B are stacked and arranged, and a partition portion 36 is also provided. A bending adjustment unit 37 is fixedly provided on the attachment surface 43pu of each attachment portion 43p by threadably connecting fixing screws 48 to the screw holes 43f through unit holes 37h.

As described above, the respective opening-side end faces 43pf of the pair of unit fixing portions 43 provided in the main frame 40 project from the one flat surface 41f. As a result, when the first chain cover 36A is mounted on the main frame 40, the first chain cover 36A is arranged between the pair of unit fixing portions 43, enabling the first chain cover 36A to be easily mounted on the one surface of the main frame 40.

Also, in a state in which the first chain cover 36A is mounted on the one flat surface 41f, the opening-side end face 43pf of each unit fixing portion 43 provided in the main frame 40 is positioned at a level that is the same as or is lower than the one flat surface 36f of the first chain cover 36A. As a result, when bending wires 32 are joined to chain joining members 38 attached to the left/right chain 35LR or the bending wires 32 are detached from the chain joining members 38, the chain joining members 38 are prevented from coming into contact with the unit fixing portions 43 when the joining members 38 mounted on the one flat surface 41f is slid and moved, enabling workability enhancement.

The present invention is not limited only to the embodiment described above, and various modifications are possible without departing from the spirit of the invention.

The present invention enables provision of an inexpensive small-size endoscope having favorable operability by manufacturing a baseplate that enables thickness reduction, by means of press working, which is inexpensive manufacturing means compared to die casting.

What is claimed is:

1. An endoscope comprising:
an insertion portion configured to be inserted into a subject;
a plate-like member provided on a proximal end side of the insertion portion, the plate-like member allowing a mechanism component for operating the insertion portion to be arranged on the plate-like member, the plate-like member having a predetermined thickness dimension;
an attachment portion including a flexed portion formed by flexing an end portion of the plate-like member in a predetermined direction;
an opening portion provided in the plate-like member, the opening portion being configured to open adjacent to the attachment portion; and
a fixing portion configured to fix at least part of another member within a range of the thickness of the plate-like member, the fixing portion being formed inside the attachment portion.

2. The endoscope according to claim 1, wherein the attachment portion is provided integrally with the flexed portion, and includes an attachment surface for abutment with the other member, the attachment surface being formed toward the predetermined direction.

3. The endoscope according to claim 1, wherein the attachment portion is arranged in a positional relationship in which the attachment portion is orthogonal to one flat surface of the plate-like member.

4. The endoscope according to claim 1, wherein:
the opening portion includes a pair of cutout portions; and
the cutout portions form a first connection portion and a second connection portion each connecting the flexed portion and the attachment portion integrally in the plate-like member.

5. The endoscope according to claim 1, further comprising an operation portion configured to operate the insertion portion, the operation portion being joined to a proximal end portion of the insertion portion, wherein the plate-like member is fixed inside the operation portion.

6. An endoscope comprising:
an insertion portion configured to be inserted into a subject;
a plate-like member provided on a proximal end side of the insertion portion, the plate-like member allowing a mechanism component for operating the insertion portion to be arranged on the plate-like member, the plate-like member having a predetermined thickness dimension;
an attachment portion formed by flexing an end portion of the plate-like member in a predetermined direction;
an end face of the attachment portion, the end face being formed toward a direction opposite to the predetermined direction and protruding in the direction opposite to the predetermined direction relative to one flat surface of the plate-like member, the one flat surface being formed toward the direction opposite to the predetermined direction; and
a fixing portion configured to fix at least part of another member within a range of the thickness of the plate-like member, the fixing portion being formed inside the attachment portion.
